(19) United States
(12) Patent Application Publication
(10) Pub. No.: US 2010/0082000 A1
(43) Pub. Date: Apr. 1, 2010

(54) CATHETER SYSTEM WITH REINFORCED MEMBER

(75) Inventors: Jordon Honeck, Maple Grove, MN (US); Ronan Thornton, County Galway (IE); Michael R. Neidert, County Galway (IE); Daniel Vinup, Maple Grove, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/241,741

(22) Filed: Sep. 30, 2008

(51) Int. Cl. A61M 25/14; A61M 25/00

(52) U.S. Cl. 604/246; 604/526

(57) ABSTRACT

A catheter system for positioning of a medical instrument within a body. The catheter system includes a hub defining a main lumen therethrough for passage of the medical instrument. The catheter system also includes an elongate tubular member defining a lumen therethrough. The lumen of the elongate tubular member is in communication with the main lumen of the hub for further passage of the medical instrument. The elongate tubular member defines a first portion and a second portion. The catheter system also includes a reinforcement member that is fixedly coupled to and reinforces the elongate tubular member, the reinforcement member includes a first part, a second part, and a partition defined between the first and second parts. The first and second parts are adapted to selectively move away from each other along the partition to thereby move the first and second portions away from each other and to uncover the medical instrument.

### FIELD

The present disclosure relates generally to medical devices and, more specifically, to a peelable catheter with a reinforced tubular member used to position an elongate medical instrument within a body.

### BACKGROUND

The statements in this section merely provide background information related to the present disclosure and may not constitute prior art.

Various devices have been proposed for introducing and/or positioning elongate medical instruments in the human body. For instance, various catheter systems, introducer sheaths, and other elongate tubular members have been proposed for these purposes. More specifically, these devices include a proximal portion that remains outside the body and a tubular member that extends into the body. A lumen extends through the proximal portion and the tubular member. A medical instrument, such as a cardiac pacemaker lead or other vascular instrument, passes through the lumen into the body, and then the medical professional positions the medical instrument into a desired position. For instance, in some embodiments, the medical instrument is positioned within the patient's heart, blood vessel, or other portion of the body. Then, the medical professional removes the tubular member of the catheter system, leaving the medical instrument in its intended position within the body.

Many catheter systems have been proposed that are designed to be removed without disturbing (i.e., inadvertently moving) the medical instrument from its desired position. For instance, in some catheter systems, the elongate tubular member can be torn and peeled apart longitudinally while being pulled out of the body, leaving the medical instrument in its intended position.

Although these conventional catheter systems have functioned for their intended purposes, problems remain. For instance, the tubular member is typically made out of a relatively flexible material so that it can be more easily peeled apart. However, when inserting and positioning the tubular member in the body, the tubular member often inadvertently flexes, bends, and twists when pushing longitudinally and/or subjecting the tubular member to torque forces. Accordingly, it can be difficult to properly position the catheter system within the body.

### SUMMARY

This section provides a general summary of the disclosure, and is not a comprehensive disclosure of its full scope or all of its features.

A catheter system is disclosed for positioning of a medical instrument within a body. The catheter system includes a hub defining a main lumen therethrough for passage of the medical instrument. The catheter system also includes an elongate tubular member defining a lumen therethrough. The lumen of the elongate tubular member is in communication with the main lumen of the hub for further passage of the medical instrument. The elongate tubular member defines a first portion and a second portion. The catheter system also includes a reinforcement member that is fixedly coupled to and reinforces the elongate tubular member, the reinforcement member includes a first part, a second part, and a partition defined between the first and second parts. The first and second parts are adapted to selectively move away from each other along the partition to thereby move the first and second portions away from each other and to uncover the medical instrument.

In another aspect, a method of positioning a medical instrument relative to a body is disclosed. The method includes positioning a portion of a catheter system relative to the body. The catheter system includes a hub with a main lumen, an elongate tubular member defining a lumen that is in communication with the main lumen, and a reinforcement member that is fixedly coupled to and reinforces the elongate tubular member. The method also includes extending the medical instrument through the main lumen and the lumen of the elongate tubular member. Furthermore, the method includes moving portions of the elongate tubular member and parts of the reinforcement member away from each other along a predetermined partition of the reinforcement member to uncover the medical instrument.

In still another aspect, a catheter system is disclosed for positioning of a medical instrument within a body. The catheter system includes a hub defining a main lumen therethrough for passage of the medical instrument. The hub includes a first piece and a second piece that is removably coupled to the first piece. The catheter system also includes an elongate tubular member defining a longitudinal axis and defining a lumen therethrough. The lumen of the elongate tubular member is in communication with the main lumen of the hub for further passage of the medical instrument. The elongate tubular member defines a first portion and a second portion. Furthermore, the catheter system includes a reinforcement member that is fixedly coupled to and reinforces the elongate tubular member. The reinforcement member includes a first part, a second part, and a partition defined between the first and second parts. The first piece is coupled to the first portion of the elongate tubular member and the first part of the reinforcement member, and the second piece is coupled to the second portion of the elongate tubular member and the second part of the reinforcement member. Movement of the first piece away from the second piece causes a slit to propagate in the elongate tubular member generally parallel to the longitudinal axis and causes the first portion to move away from the second portion and the first part to move away from the second part.

Further areas of applicability will become apparent from the description provided herein. The description and specific examples in this summary are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of a catheter system according to various teachings of the present disclosure;

FIG. 2 is an exploded perspective view of the catheter system of FIG. 1;

FIG. 3 is a section view of the catheter system taken along the line 3-3 of FIG. 1;

FIG. 4 is a section view of the catheter system taken along the line 4-4 of FIG. 1;

FIG. 5 is a perspective view of the catheter system of FIG. 1, shown in a state of peeling a tubular member apart;

FIG. 6 is a perspective view of the tubular member of the catheter system of FIG. 1;

FIG. 7 is a perspective view of a reinforcement member of the catheter system of FIG. 1;

FIG. 8 is a perspective view of another embodiment of the reinforcement member;

FIG. 9 is a perspective view of another embodiment of the reinforcement member;

FIG. 10 is a perspective view of another embodiment of the reinforcement member;

FIG. 11 is a perspective view of another embodiment of the reinforcement member;

FIG. 12 is a perspective view of another embodiment of the reinforcement member;

FIG. 13 is a perspective view of another embodiment of the reinforcement member;

FIG. 14 is a perspective view of another embodiment of the reinforcement member;

FIG. 15 is a perspective view of another embodiment of the reinforcement member; and

FIG. 16 is a perspective view of the catheter system according to various teachings of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

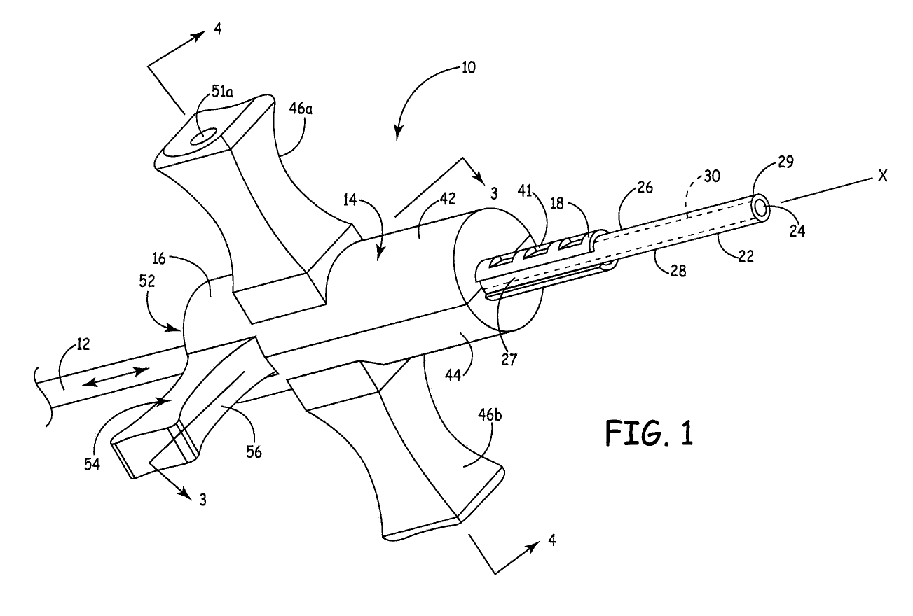

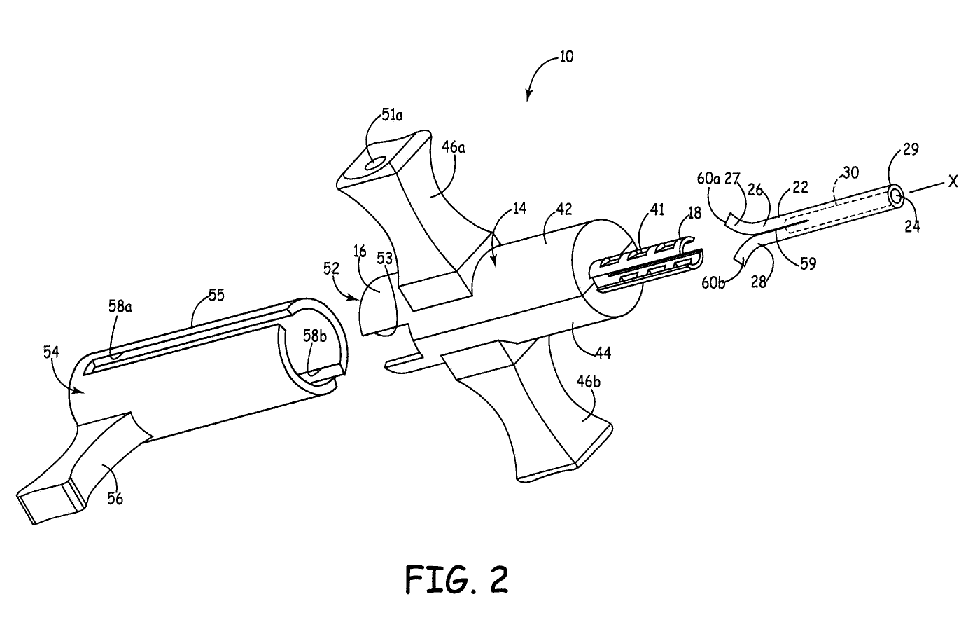

Referring initially to FIGS. 1 and 2, a catheter system 10 is illustrated. The catheter system 10 is operable for positioning an elongate medical instrument 12, such as a cardiac pacemaker lead, vascular instrument, a suture sleeve, or other elongate medical instrument, within a body of a patient. It will be appreciated that, although the following discussion will be related to positioning a cardiac pacemaker or other vascular instrument, the catheter system 10 can be used for positioning any suitable medical instrument 12 without departing from the scope of the present disclosure.

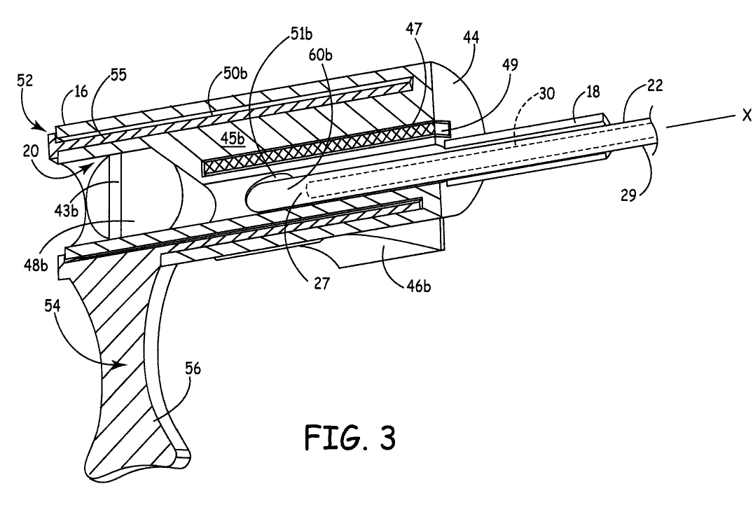

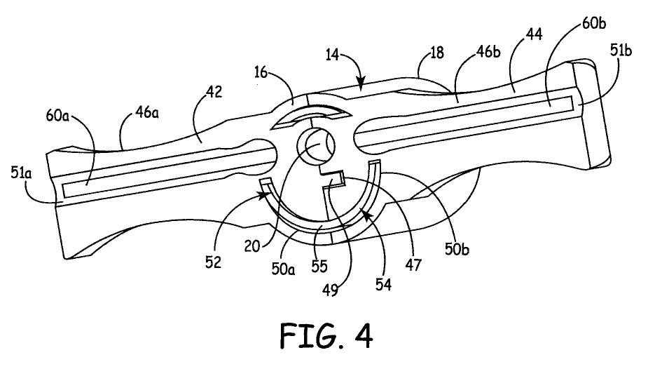

As shown in FIGS. 1 through 5, the catheter system 10 includes a hub 14 having a proximal end 16, a distal end 18, and a main lumen 20 (FIGS. 3 and 4) that extends between the proximal and distal ends 18. In some embodiments, the main lumen 20 defines an axis X of the hub 14. As will be described in greater detail below, the medical instrument 12 passes through the main lumen 20 for positioning of the medical instrument 12.

Furthermore, the catheter system 10 includes an elongate tubular member 22 defining a lumen 24 therethrough. The elongate tubular member 22 shares the axis X with the hub 14. The elongate tubular member 22 includes a proximal end 27 and a distal end 29. The proximal end 27 is coupled to the hub 14 in a manner to be discussed in greater detail below. The lumen 24 is in communication with the main lumen 20 of the hub 14. As will be discussed, the medical instrument 12 passes through the main lumen 20 and further passes through the lumen 24 and into the body.

Furthermore, the tubular member 22 defines a first portion 26 and a second portion 28. The first and second portions 26, 28 are each elongate, and each has an arcuate cross section. The first and second portions 26, 28 are disposed substantially symmetrically about the axis X such that the tubular member 22 is hollow and tubular. The first and second portions 26, 28 are operably secured so as to cooperatively define the lumen 24. In some embodiments, the first and second portions 26, 28 are integrally coupled such that the tubular member 22 is monolithic. As will be discussed in greater detail, the first and second portions 26, 28 are operable to move away from each after the medical instrument 12 has been positioned in the body in order to uncover the medical instrument 12 and to remove the catheter system 10.

Moreover, the catheter system 10 includes a reinforcement member 30, which is shown in phantom in FIGS. 1, 2, and 3. In some embodiments, the reinforcement member 30 is elongate and generally tubular and shares the axis X with the hub 14 and tubular member 22. The reinforcement member 30 is fixedly coupled to the tubular member 22. In some embodiments, the reinforcement member 30 is disposed between an outer sheath 32 and an inner sheath 34 of the tubular member 22 (FIG. 6). For instance, in order to join the reinforcement member 30 and the tubular member 22 in some embodiments, the reinforcement member 30 is disposed between separate inner and outer sheaths 32, 34, and then the inner and outer sheaths 32, 34 are hot melted together with the reinforcement member 30 embedded therebetween. Furthermore, in some embodiments, the reinforcement member 30 is overmolded to the tubular member 22. It will be appreciated that the reinforcement member 30 could be fixed to the tubular member 22 in any suitable fashion.

Accordingly, as will be discussed, the reinforcement member 30 reinforces the tubular member 22 such that the tubular member 22 resists kinking and such that torque forces transfer effectively through to the tubular member 22 while positioning the catheter system 10 relative to the body. In some embodiments, the reinforcement member 30 allows approximately 1:1 torque transfer from the proximal end 27 to the distal end 29. Accordingly, the catheter system 10 can be more quickly and easily positioned relative to the body.

In addition, the reinforcement member 30 includes a first part 36 and a second part 38 (FIGS. 6 and 7). In some embodiments, the first and second parts 36, 38 are elongate, and each has a substantially arcuate cross section. The first and second parts 36, 38 are disposed substantially symmetrically about the axis X such that the reinforcement member 30 is generally hollow and tubular. Also, in some embodiments represented in FIGS. 6 and 7, the first and second parts 36, 38 are disposed at a distance away from each other in a direction transverse to the axis X so as to define a partition 40. In some embodiments, the reinforcement 30 includes a plurality of partitions 40 that each extend generally parallel to the longitudinal axis X, and the partitions 40 are disposed substantially symmetrically on opposite sides of the axis X.

The first part 36 of the reinforcement member 30 is fixed to the first portion 26 of the tubular member 22, and the second part 38 is fixed to the second portion 28 of the tubular member 22. As will be described, the first and second parts 36, 38 are adapted to selectively move away from each other and part along the partitions 40 to thereby move the first and second portions 26, 28 away from each other once the medical instrument 12 has been positioned in the body. Thus, the reinforcement member 30 and tubular member 22 can peel apart, separate, break, sever, or split generally along the longitudinal axis X to uncover the medical instrument 12. More specifically, as will be described, the reinforcement member 30 and tubular member 22 can peel apart or separate due to slits that propagate generally parallel to the longitudinal axis X.

Various embodiments of the hub 14 will now be discussed in greater detail. The hub 14 includes a first piece 42 and a second piece 44 that are removably coupled to each other. The first piece 42 and second piece 44 are substantially symmetric with respect to the axis X. In some embodiments, the first and second pieces 42, 44 are made out of a polymeric, molded material, such as PEBAX. Also, the first and second pieces 42, 44 each have a generally arcuate cross section and include a handle 46a, 46b extending transverse and symmetrically away from the axis. Furthermore, the first and second pieces 42, 44 include a plurality of apertures 41 at the distal end 18 thereof, which provide added flexibility to the distal end 18 and which provide a relatively smooth transition from the hub 14 to the tubular member 22 so as to reduce the possibility of kinking of the tubular member 22 due to pushing the hub 14 along the axis X. Also, the first and second pieces 42, 44 include a respective abutment surface 45a, 45b, which are substantially flat so as to abut and mate with each other.

In some embodiments, the first and second pieces 42, 44 include a hydrophobic material (not specifically shown) for reducing leakage from the hub 14. For instance, the pieces 42, 44 can be constructed at least partially from hydrophobic material, such as PE or PTFE. The pieces 42, 44 can also be coated on the abutment surfaces 45a, 45b with a hydrophobic coating, such as silicone. Also, the entire hub 14 can be coated with a thin, continuous, low-tear strength material such that leaking is reduced and yet the pieces 42, 44 can be easily removed from each other.

Also, the first and second pieces 42, 44 each include a recess 48a, 48b that cooperate to define the main lumen 20 of the hub 14. In some embodiments, the recesses 48a, 48b are shaped such that the main lumen 20 is tapered. More specifically, in some embodiments, the main lumen 20 tapers such that the main lumen 20 is wider adjacent the proximal end 16 as compared to the distal end 18.

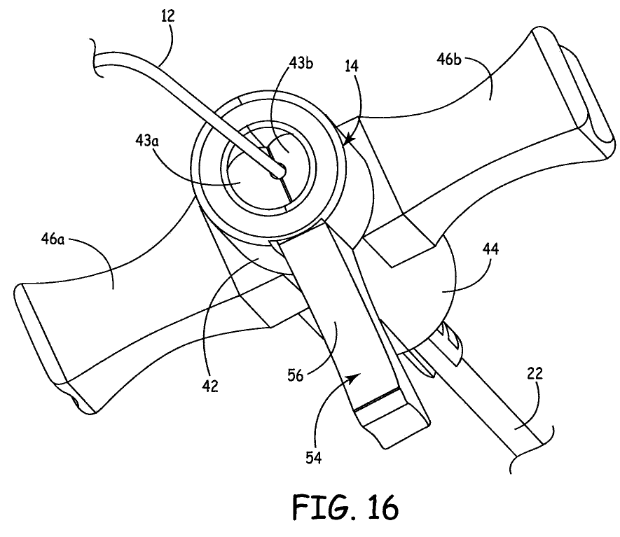

As shown in FIGS. 3 and 16, the first piece 42 can include a first valve portion 43a, and the second piece 44 can include a second valve portion 43b. The first valve portion 43a extends toward the axis X from within the recess 48a, and the second valve portion 43b extends toward the axis X from within the recess 48b. As shown in FIG. 16, the valve portions 43a, 43b operatively engage each other when the first and second pieces 42, 44 are operatively secured. Thus, the valve portions 43a, 43b can regulate flow through the main lumen 20. More specifically, in some embodiments, the valve portions 43a, 43b are resiliently flexible to allow the instrument 12 to pass between the valve portions 43a, 43b; however, the valve portions 43a, 43b each seal against the medical instrument 12 and against each other to substantially reduce backflow through the main lumen 20 and out of the hub 14 away from the tubular member 22.

In some embodiments illustrated in FIGS. 3, 4, and 5, the first piece 42 includes a tongue 49 that extends substantially parallel to the axis X and away from the respective abutment surface 45a, and the second piece 44 includes a groove 47 that extends substantially parallel to the axis X and away from the respective abutment surface 45b. The groove 47 receives the tongue 49 to further couple the first and second pieces 42, 44.

In addition, in some embodiments illustrated in FIGS. 1, 2, 4, and 5, the first and second pieces 42, 44 each include a secondary lumen 51a, 51b. In some embodiments, the secondary lumens 51a, 51b each extend through a respective one of the handles 46a, 46b and are each in communication with the main lumen 20. As will be described in greater detail, the elongate tubular member 22 extends into the secondary lumens 51a, 51b to be operably secured to the hub 14 in some embodiments. More specifically, the first piece 42 is operably coupled to the first portion 26 of the tubular member 22 and the first part 36 of the reinforcement member 30, and the second piece 44 is operably coupled to the second portion 28 of the tubular member 22 and the second part 38 of the reinforcement member 30 such that selective movement of the first piece 42 of the hub 14 away from the second piece 44 causes the first portion 26 of the tubular member 22 to move away from the second portion 28 and causes the first part 36 of the reinforcement member 30 to move away from the second part 38. Thus, as will be discussed, peeling the catheter system 10 apart can be completed quickly and easily, and the medical instrument 12 is unlikely to be inadvertently moved during this peeling process.

Furthermore, in some embodiments, the first and second pieces 42, 44 each define a rounded slot 50a, 50b that extend generally along the axis X. The slots 50a, 50b cooperate to define an opening 52 that extends partially along the length of the hub 14 from the proximal end 16. The opening 52 substantially encompasses the main lumen 20. The opening 52 removably receives a retainer member 54, which removably couples the first and second piece 42, 44.

As best shown in FIGS. 2 and 3, the retainer member 54 includes a hollow tubular portion 55 that shares the axis X. Also, the retainer member 54 includes a handle 56 that is operably secured to the tubular portion 55 and that extends transversely away from the axis X. In some embodiments, the retainer member 54 is made out of molded, polymeric material, such as PEBAX. The tubular portion 55 includes a plurality of notches 58a, 58b (FIG. 2) that are disposed symmetrically about the axis X and that are disposed on opposite sides of the handle 56.

The retainer member 54 is moveable longitudinally along the X axis into and out of the opening 52. More specifically, the tubular portion 55 is moveably received in the opening 52. When the tubular portion 55 is disposed in the opening 52, the tubular portion 55 removably couples the first and second pieces 42, 44. Also, the tubular portion 55 can be selectively removed from the opening 52 generally along the X axis in order to decouple the first and second pieces 42, 44.

The notches 58a, 58b in the tubular portion 55 are included in order to avoid interference with the handles 46a, 46b. Also, in some embodiments, the first and second pieces 42, 44 cooperate to define a slot 53 at the proximal end 16 of the hub 14. The slot 53 receives the handle 56 of the retainer member 54 when the retainer member 54 is disposed in the opening 52.

Accordingly, the retainer member 54 securely couples the first and second pieces 42, 44. Also, the retainer member 54 can be easily removed from the opening 52 for facilitating decoupling of the first and second pieces 42, 44. It will be appreciated, however, that the first and second pieces 42, 44 could be secured in any suitable fashion, with or without the retainer member 54. For instance, the first and second pieces 42, 44 can be operably secured with an adhesive, in addition to or instead of the retainer member 54. Also, in some embodiments, the first and second pieces 42, 44 could be retained with clips, pins, and otherwise without departing from the scope of the present disclosure.

Now, various embodiments of the tubular member 22 will be discussed in greater detail. In some embodiments, the tubular member 22 has a longitudinal length that is greater than the longitudinal length of the reinforcement member 30. For instance, in some embodiments, the distal end 29 of the tubular member 22 extends longitudinally away from a corresponding end of the reinforcement member 30. In some embodiments, the distal end 29 is made longer than that of the reinforcement member 30 by dipping the tubular member 22 and reinforcement member 30 into a bath of polymeric material that adheres to the distal end 29. Accordingly, the distal end 29 is relatively soft, and the distal end 29 can be more easily moved through the body. Also, in some embodiments, the distal end 29 can include a material that highly visible in X-ray or other imaging methods. Furthermore, in some embodiments, the proximal end 27 of the tubular member 22 extends away from a corresponding end of the reinforcement member 30. As such, the proximal end 27 of the tubular member 22 can be more easily coupled to the hub 14 as will be described.

Specifically, as shown in the embodiment of FIG. 2, the proximal end 27 extends away from the corresponding end of the tubular member 22, and the proximal end 27 includes a predefined slit 59. In the embodiment illustrated, the proximal end 27 includes a plurality of slits 59 that extend longitudinally and that are disposed on opposite sides of the axis X. In the embodiment shown in FIG. 2, there are two slits 59, one of which is shown, and the other of which is disposed symmetrically with respect to the axis X on an opposite side of the axis X. The slits 59 are also disposed between the first and second portions 26, 28 of the tubular member 22 and also between the first and second parts 36, 38 of the reinforcement member 30. In some embodiments, the slits 59 are approximately one to two centimeters in length.

Accordingly, the proximal end 27 defines a plurality of tabs 60a, 60b. Each tab 60a, 60b extends into a corresponding one of the secondary lumens 51a, 51b as shown in FIG. 4. Each tab 60a, 60b is also operably secured to the one of the first and second pieces 42, 44 on the corresponding inner surface of the secondary lumen 51a, 51b. In some embodiments, the tabs 60a, 60b are attached to the hub 14 via an adhesive. For instance, in some embodiments, cyanoacrylate (CA) adhesive is introduced into the secondary lumens 51a, 51b to operably secure the tabs 60a, 60b to the hub 14. However, it will be appreciated that the tabs 60a, 60b could be attached in any suitable manner without departing from the scope of the present disclosure. It will be appreciated that the secondary lumens 51a, 51b allow for ease of attaching the tubular member 22 to the hub 14. It will be appreciated, however, that the secondary lumens 51a, 51b could also be used for the flow of fluid into and/or out of the main lumen 20. For instance, dye or other suitable fluid could be introduced into the main lumen 20 and into the patient through the secondary lumens 51a, 51b.

In some embodiments, other portions of the tubular member 22 are also coupled to the hub 14. For instance, in some embodiments, a portion of the tubular member 22 forward of the slit 59 is coupled to the hub 14 inside the main lumen 20 to further secure the hub 14 and tubular member 22. Also, in some embodiments, the hub 14 can be overmolded onto the tubular member 22 for operably securing the hub 14 and tubular member 22.

Also, as the first and second pieces 42, 44 of the hub 14 move away from each other, the predefined slit 59 propagates longitudinally along the tubular member 22 in a direction generally parallel to the axis X in order to reveal the medical instrument 12. It will be appreciated that the slits 59 facilitate peeling or separating of the tubular member 22 along the axis X. As such, the tubular member 22 can be easier to peel apart, and the tubular member 22 is more likely to peel relatively straight in a direction substantially parallel to the axis. Accordingly, the medical instrument 12 is less likely to be disturbed during peeling of the tubular member 22. It will be appreciated that the tubular member 22 could also be scored between the first and second portions 26, 28 with scoring (not shown) that meets the slits 59 to further facilitate peeling and to further define a predetermined failure mode.

Thus, referring initially to FIG. 1, an exemplary procedure for using of the catheter system 10 will be discussed. The tubular member 22 can be inserted into the body (not shown) through a prepared incision (not shown). In some embodiments, a pull wire (not shown) extends from the distal end 29 to the proximal end 16 of the hub 14, and by pulling on the pull wire, the user can deflect the tubular member 22 for proper positioning of the tubular member 22.

Next, the medical instrument 12 is inserted by passing between the valve portions 43a, 43b and through the main lumen 20. The medical instrument 12 then further passes through the lumen 24 of the tubular member 22 into the body.

Then, the user grasps the handle 56 of the retainer member 54 with one hand and the hub 14 with the other and pulls the retainer member longitudinally away from the proximal end 16 of the hub 14. The retainer member 54 can then be discarded.

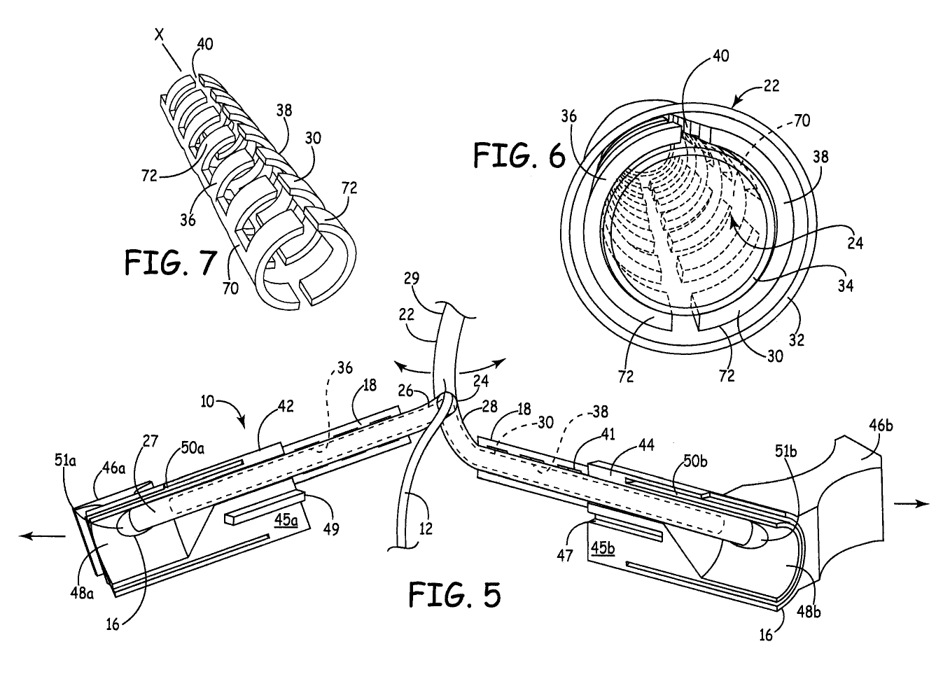

Next, the user grasps the handles 46a, 46b and pulls the first and second pieces 42, 44 away from each other away from the axis X as shown in FIG. 5. This movement coincidentally peels the first and second portions 26, 28 of the tubular member 22 and the first and second parts 36, 38 of the reinforcement member 30 away from each other. As the tubular member 22 peels apart, the tubular member and reinforcement member 30 are pulled from the patient's body, leaving the medical instrument 12 in its intended position.

Accordingly, the catheter system 10 allows for easy positioning of the medical instrument 12. Also, the catheter system 10 can be easily positioned and removed from the body and is unlikely to disturb the medical instrument 12 from its intended position. Also, the system 10 can be positioned and removed by one person because of the various features described above. In addition, manufacture of the catheter system 10 can be completed relatively quickly and less expensively than systems of the prior art.

Moreover, as stated above, the reinforcement member 30 reinforces the tubular member 22 such that the tubular member 22 is sufficiently flexible, but yet is sufficiently stiff to allow pushing and/or torque forces to be transferred effectively through the tubular member 22. Thus, the tubular member 22 can be pushed into, twisted, and generally positioned within the body quickly and easily.

Various embodiments of the reinforcement member 30 will now be discussed with reference to FIGS. 6 through 15. Components that are related will be identified with corresponding reference numerals differing by intervals of 100.

In the embodiments represented in FIGS. 6 and 7, the first and second parts 36, 38 of the reinforcement member 30 each include a spine element 70. In some embodiments, the spine element 70 is substantially continuous and extends generally parallel to the longitudinal axis. Moreover, the reinforcement member 30 includes a plurality of ribs 72. Each rib 72 extends transversely from a side of the respective spine element 70 and is rounded about the axis X. Also, the ribs 72 are disposed in spaced relationship from each other along the axis X. The partition 40 of the reinforcement member 30 is defined generally between the terminal ends of the ribs 72. Accordingly, the reinforcement member 30 provides sufficient stiffness to the tubular member 22 and yet still allows for sufficient flexibility.

In some embodiments represented in FIG. 9, the width of the spine element 170 changes along the longitudinal axis X. More specifically, in some embodiments, the spine element 170 includes a tapered portion 172. As such, the spine element 170 is wider adjacent the proximal end 127 as compared to the spine element 170 adjacent the distal end 129. Thus, the properties of the tubular member 22 can be varied along the axis X. For instance, the spine element 170 can be thicker adjacent the proximal end 27 of the tubular member 22 to better transfer pushing forces, and the spine element 170 can include the tapered portion 172 adjacent the distal end 29 to make the tubular member softer adjacent the distal end 29.

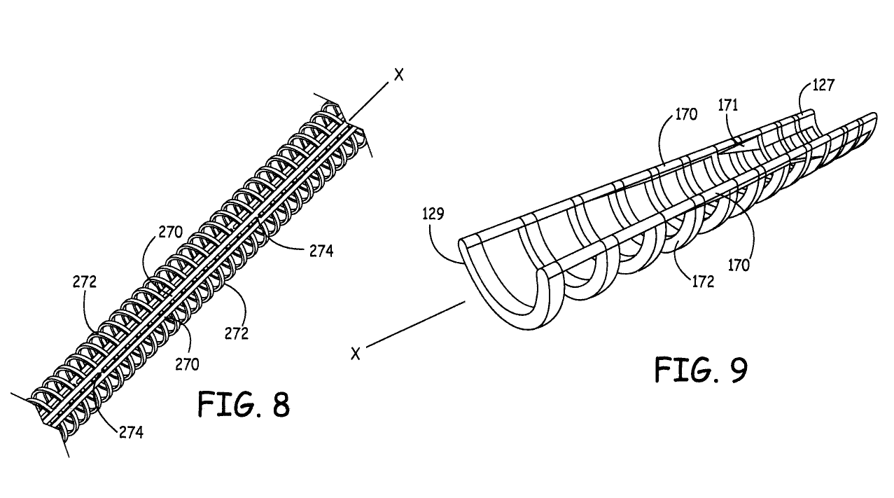

Furthermore, in some embodiments represented in FIG. 8, the first and second parts 236, 238 are removably coupled, independent of the tubular member 222. For instance, in some embodiments, the reinforcement member 230 includes a breakable joint 274 that removably couples the first and second parts 236, 238. As shown, the breakable joint 274 extends generally across the respective partition 240, transverse to the axis X, and has a thickness that is significantly less than that of the other portions of the reinforcement member 230. Thus, during peeling action, the breakable joints 274 consecutively break to allow the first and second parts 236, 238 to move away from each other.

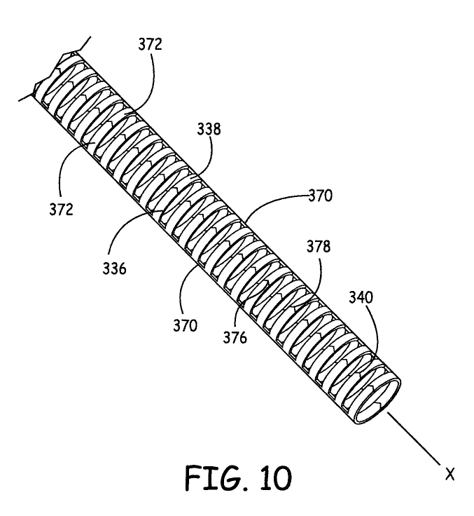

Alternatively, in some embodiments represented in FIG. 10, the reinforcement member 330 includes a plurality of recesses 378 that removably receive corresponding projections 376 to thereby removably engage the first and second parts 336, 338 of the reinforcement member 330. For instance, in some embodiments, represented in FIG. 10, the ribs 372 of the first part 336 each include a tapered projection 376 at the terminal end thereof, and the ribs 372 of the second part 338 each include a corresponding tapered recess 378 at the terminal end thereof. As such, the projections and recesses 376, 378 extend in a direction generally transverse to the axis X. As shown, the recesses 378 each removably receive a corresponding projection 376. It will be appreciated that the partition 340 is defined between the corresponding pairs of projections 376 and recesses 378. Because of the shape of the projections 376 and recesses 378, the reinforcement member 330 can transfer forces more effectively in a plurality of directions for more effective reinforcement of the tubular member 22.

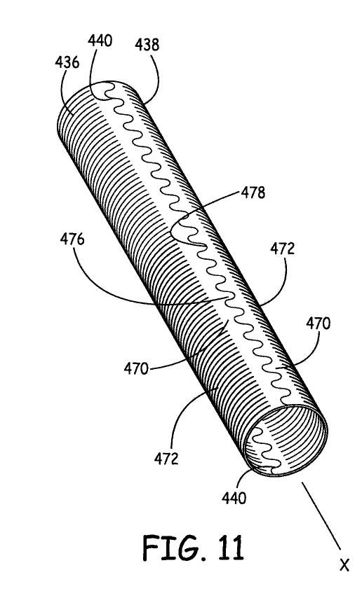

Also, in some embodiments represented in FIG. 11, the spine elements 470 are defined at each terminal end of the first and second parts 436, 438. The partitions 440 are defined between each pair of spine elements 470. Also, the spine elements 470 are substantially sinusoidal in shape so as to define a plurality of rounded projections 476 and a plurality of correspondingly rounded recesses 478. Accordingly, the projections 476 and recesses 478 are alternatingly arranged on each spine element 470. The recesses 478 removably receive the projections 476 to thereby removably engage the first and second parts 436, 438 of the reinforcement member 430.

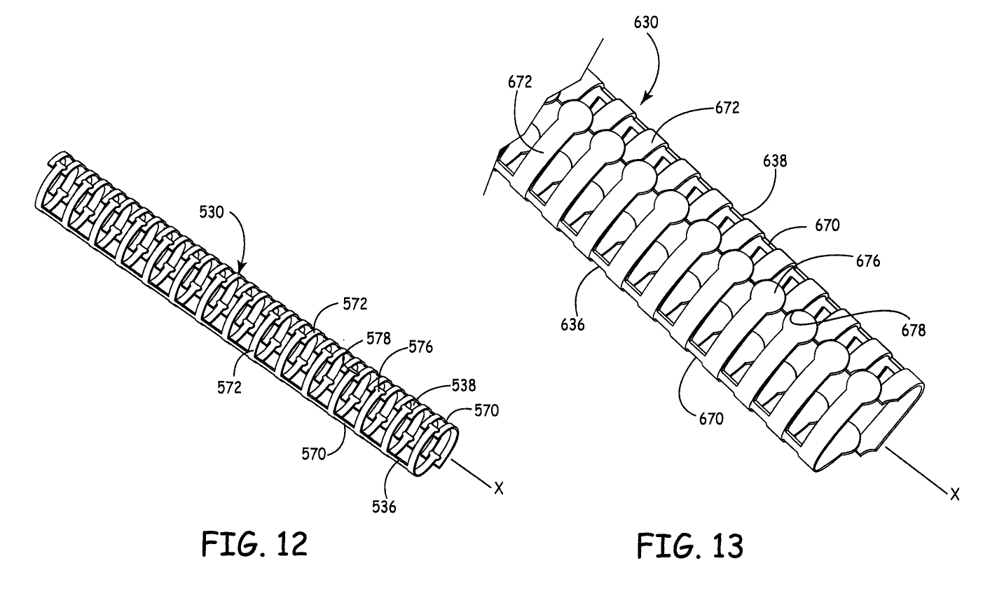

In addition, in some embodiments represented in FIG. 12, the first part 536 includes a plurality of ribs 572 that extend transversely from corresponding ribs 570, and the projections 576 extend outward from the terminal end of the ribs 570 in a direction substantially parallel to the axis X. The projections 576 are substantially rounded. Also, the ribs 572 of the second part 538 include recesses 578 that extend substantially parallel to the axis X and are correspondingly rounded. Accordingly, the recesses 578 receive corresponding projections 576 in order to removably engage the first and second parts 536, 538 of the reinforcement member 530.

Still further, in some embodiments represented in FIG. 13, the first part 636 includes a plurality of circular, rounded projections 676, and the terminal end of the ribs 672 of the second part 638 include partially circular recesses 678. The projections 676 are disposed between corresponding pairs of ribs 672 and are received in corresponding recesses 678 to thereby removably engage the first and second parts 636, 638 of the reinforcement member 630.

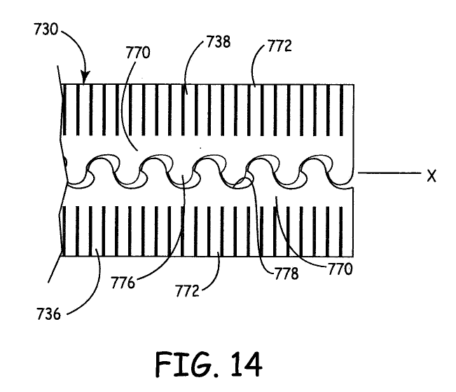

In addition, FIG. 14 illustrates another embodiment of the reinforcement member 730, which is substantially similar to the reinforcement member 430 of FIG. 11. However, in the embodiment of FIG. 14, the projections 776 and recesses 778 extend partially along the axis X. Accordingly, during peeling, the projections 776 cam against the surfaces of the corresponding recesses 778 to facilitate peeling motion.

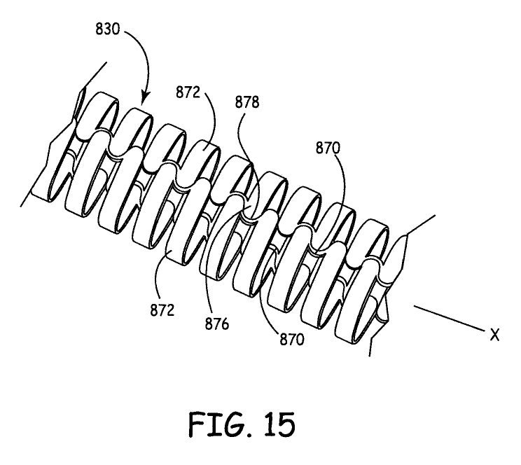

Furthermore, in some embodiments represented in FIG. 15, the terminal ends of the ribs 872 are each rounded to define a plurality of projections 876. Also, the spine elements 870 are rounded so as to define a plurality of recesses 878. In addition, the spine elements 870 extend between corresponding pairs of the ribs 872, and the spine elements 870 are disposed in alternating arrangement on opposite sides of the axis X along the longitudinal length of the reinforcement member 830. Accordingly, the location of the spine elements 870 alternates along the axis X. The projections 876 are received within corresponding recesses 878.

Accordingly, each of the reinforcement members 30, 130, 230, 330, 430, 530, 630, 730, 830 reinforces the tubular member 22 with sufficient resiliency and flexibility, and allows pushing, torque and other forces to effectively transfer through the tubular member 22. Thus, the tubular member 22 can be positioned within the body more easily. Also, the reinforcement members 30, 130, 230, 330, 430, 530, 630, 730, 830 can be easily parted in order to peel the tubular member 22 for removal of the catheter system 10.

Certain terminology is used herein for purposes of reference only, and thus is not intended to be limiting. For example, terms such as “upper,” “lower,” “above,” “below,” “top,” “upward,” and “downward” refer to directions in the drawings to which reference is made. Terms such as “front,” “back,” “rear,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features and the exemplary embodiments, the articles “a,” “an,” “the” and “said” are intended to mean that there are one or more of such elements or features. The terms “comprising,” “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A catheter system for positioning of a medical instrument within a body, the catheter system comprising:
a hub defining a main lumen therethrough for passage of the medical instrument;
an elongate tubular member defining a lumen therethrough, the lumen of the elongate tubular member being in communication with the main lumen of the hub for further passage of the medical instrument, the elongate tubular member defining a first portion and a second portion; and
a reinforcement member that is fixedly coupled to and reinforces the elongate tubular member, the reinforcement member including a first part, a second part, and a partition defined between the first and second parts, the first and second parts adapted to selectively move away from each other along the partition to thereby move the first and second portions away from each other and to uncover the medical instrument.

2. The catheter system of claim 1, wherein the elongate tubular member includes an outer sheath and an inner sheath, the reinforcement member fixedly coupled to the elongate tubular member between the outer and inner sheaths.

3. The catheter system of claim 1, wherein the elongate tubular member and the reinforcement member substantially share a common longitudinal axis, wherein the first and second portions of the elongate tubular member are operable to peel away from each other by a slit that propagates generally parallel to the longitudinal axis, and wherein the first and second parts of the reinforcement member are operable to peel away from each other by the slit that propagates generally parallel to the longitudinal axis.

4. The catheter system of claim 1, wherein the first and second parts of the reinforcement member are disposed at a distance away from each other to define the partition.

5. The catheter system of claim 1, wherein the first and second parts of the reinforcement member are removably coupled, independent of the elongate tubular member.

6. The catheter system of claim 5, wherein the reinforcement member includes a breakable joint that removably couples the first and second parts, the joint spanning across the partition.

7. The catheter system of claim 6, wherein the reinforcement member defines a longitudinal axis, wherein the partition extends generally parallel to the longitudinal axis, and wherein the breakable joint extends generally transverse to the longitudinal axis.

8. The catheter system of claim 5, wherein one of the first and second parts of the reinforcement member includes a projection and the other of the first and second parts includes a recess that receives the projection to thereby removably engage the first and second parts.

9. The catheter system of claim 8, wherein the first and second parts each include a plurality of projections and recesses, which are alternatingly arranged, and wherein the plurality of recesses receive corresponding projections to thereby removably engage the first and second parts.

10. The catheter system of claim 8, wherein the reinforcement member defines a longitudinal axis, and wherein the projection and recess extend generally transverse to the longitudinal axis.

11. The catheter system of claim 8, wherein the reinforcement member defines a longitudinal axis, and wherein the projection and recess extend substantially parallel to the longitudinal axis.

12. The catheter system of claim 8, wherein the projection is substantially rounded, and the recess is correspondingly rounded.

13. The catheter system of claim 8, wherein the projection is tapered, and the recess is correspondingly tapered.

14. The catheter system of claim 1, wherein the reinforcement member defines a longitudinal axis and includes a plurality of ribs that extend generally transverse to the longitudinal axis, the plurality of ribs separated at a distance from each other.

15. The catheter system of claim 1, wherein the reinforcement member defines a longitudinal axis and includes a spine element that extends generally parallel to the longitudinal axis.

16. The catheter system of claim 15, wherein each of the first and second parts of the reinforcement member include a spine element, wherein the partition is defined between the spine elements of the first and second parts.

17. The catheter system of claim 16, wherein the reinforcement member includes a plurality of spine elements and a plurality of ribs, wherein the spine elements extend between corresponding pairs of the ribs, and wherein the spine elements are disposed in alternating arrangement on opposite sides of a longitudinal axis of the reinforcement member.

18. The catheter system of claim 15, wherein a width of the spine element changes along the longitudinal axis.

19. The catheter system of claim 18, wherein a proximal end of the at least a portion of the spine element is wider than a distal end of the spine element.

20. The catheter system of claim 1, wherein the reinforcement member defines a longitudinal axis, wherein the reinforcement member defines a first partition and a second partition extending generally parallel to the longitudinal axis, and wherein the first and second partitions are disposed substantially symmetrically on opposite sides of the longitudinal axis.

21. The catheter system of claim 1, wherein the reinforcement member defines a longitudinal length that is less than a longitudinal length of the elongate tubular member.

22. The catheter system of claim 1, wherein the hub includes a first piece and a second piece that is removably coupled to the first piece, the first piece coupled to the first portion of the elongate tubular member and the first part of the reinforcement member, the second piece coupled to the second portion of the elongate tubular member and the second part of the reinforcement member, wherein movement of the first piece away from the second piece causes the first portion to move away from the second portion and the first part to move away from the second part.

23. The catheter system of claim 22, wherein the first piece includes a first valve portion, and the second piece includes a second valve portion, the first and second valve portions operably engageable to allow passage of the medical instrument therebetween and to reduce fluid flow in a direction through the main lumen.

24. The catheter system of claim 1, wherein the hub includes a handle that extends generally transverse to a longitudinal axis defined by the main lumen of the hub.

25. The catheter system of claim 24, wherein the handle defines a secondary lumen extending therethrough, the secondary lumen in communication with the main lumen of the hub.

26. The catheter system of claim 25, wherein the elongate tubular member extends at least partially through the main lumen of the hub and at least partially into the secondary lumen and couples to the hub within the secondary lumen.

27. The catheter system of claim 25, wherein the hub includes a first piece and a second piece that is removably coupled to the first piece, the first piece including a first handle and the second piece including a second handle, the first and second handles each extending generally transverse to the longitudinal axis, the first and second handles extending substantially symmetrically from opposite sides of the longitudinal axis, the first and second handles each defining a secondary lumen in communication with the main lumen of the hub.

28. The catheter system of claim 1, wherein the hub includes a first piece, a second piece, and a removable retainer member that removably couples the first and second piece.

29. The catheter system of claim 28, wherein the first and second piece cooperate to define an opening that receives the removable retainer member to removably couple the first and second piece.

30. The catheter system of claim 29, wherein the main lumen defines a longitudinal axis, and wherein the removable retainer is movable out of the opening generally along the longitudinal axis, thereby de-coupling the first and second piece.

31. The catheter system of claim 1, wherein the elongate tubular member includes a predefined slit at a proximal end thereof.

32. The catheter system of claim 1, wherein the hub includes a first piece and a second piece that is removably coupled to the first piece, and wherein one of the first and second piece includes a tongue and the other of the first and second piece includes a groove that receives the tongue.

33. A method of positioning a medical instrument relative to a body comprising:
positioning a portion of a catheter system relative to the body, the catheter system including a hub with a main lumen, an elongate tubular member defining a lumen that is in communication with the main lumen, and a reinforcement member that is fixedly coupled to and reinforces the elongate tubular member;
extending the medical instrument through the main lumen and the lumen of the elongate tubular member; and
moving portions of the elongate tubular member and parts of the reinforcement member away from each other along a predetermined partition of the reinforcement member to uncover the medical instrument.

34. The method of claim 33, further comprising moving pieces of the hub away from each other to thereby move portions of the elongate tubular member and parts of the reinforcement member away from each other along a predetermined partition of the reinforcement member to uncover the medical instrument.

35. A catheter system for positioning of a medical instrument within a body, the catheter system comprising:
a hub defining a main lumen therethrough for passage of the medical instrument, the hub including a first piece and a second piece that is removably coupled to the first piece;
an elongate tubular member defining a longitudinal axis and defining a lumen therethrough, the lumen of the elongate tubular member being in communication with the main lumen of the hub for further passage of the medical instrument, the elongate tubular member defining a first portion and a second portion; and
a reinforcement member that is fixedly coupled to and reinforces the elongate tubular member, the reinforcement member including a first part, a second part, and a partition defined between the first and second parts, the first piece coupled to the first portion of the elongate tubular member and the first part of the reinforcement member, the second piece coupled to the second portion of the elongate tubular member and the second part of the reinforcement member, wherein movement of the first piece away from the second piece causes a slit to propagate in the elongate tubular member generally parallel to the longitudinal axis and causes the first portion to move away from the second portion and the first part to move away from the second part.
